(19) United States
(12) Patent Application Publication
(10) Pub. No.: US 2003/0036699 A1
(43) Pub. Date: Feb. 20, 2003

(54) Methods and systems which use annexin for bioprofiling body lumen

(75) Inventor: H. William Strauss, Redwood City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IMETRx, Inc., Mountain View, CA

(21) Appl. No.: 10/080,529

(22) Filed: Feb. 21, 2002

Related U.S. Application Data
(60) Provisional application No. 60/270,884, filed on Feb. 21, 2001.

(51) Int. Cl. A61B 6/00

(52) U.S. Cl. 600/436

(57) ABSTRACT

Methods, devices, and kits, for assessing luminal lesions rely on intraluminal detection of labeled annexin. Annexin coupled to a detectable marker is introduced to a body lumen, either into the lumen itself or systemically in patient circulation, and localizes at a lesion. The detector is introduced into the body lumen and the localize distribution of the detectable marker is detected in situ. The information is useful for a number of purposes including the identification of unstable plaque in patients suffering from atherosclerotic disease.

### CROSS-REFERENCES TO RELATED APPLICATIONS

This application claims the benefit of prior provisional application No. 60/270,884, filed on Feb. 21, 2001, under 37 CFR §1.78(a)(3), the full disclosure of which is incorporated herein by reference.

### BACKGROUND OF THE INVENTION

1. Field of the Invention

The present invention relates generally to medical devices and methods. More particularly, the present invention relates to nuclear radiology and devices and methods for the intraluminal characterization of lesions in blood vessels and other body lumens.

Coronary artery disease resulting from the build-up of atherosclerotic plaque in the coronary arteries is a leading cause of death in the United States and worldwide. The plaque build-up causes a narrowing of the artery, commonly referred to as a lesion, which reduces blood flow to the myocardium (heart muscle tissue). Myocardial infarction (better known as a heart attack) can occur when an arterial lesion abruptly closes the vessel, causing complete cessation of blood flow to portions of the myocardium. Even if abrupt closure does not occur, blood flow may decrease resulting in chronically insufficient blood flow which can cause significant tissue damage over time.

A variety of interventions have been proposed to treat coronary artery disease. For disseminated disease, the most effective treatment is usually coronary artery bypass grafting where problematic lesions in the coronary arteries are bypassed using external grafts. In cases of less severe disease, pharmaceutical treatment is often sufficient. Finally, focal disease can often be treated intravascularly using a variety of catheter-based approaches, such as balloon angioplasty, atherectomy, radiation treatment, stenting, and often combinations of these approaches.

With the variety of treatment techniques which are available, the cardiologist is faced with a challenge of selecting the particular treatment which is best suited for an individual patient. While numerous of diagnostic aids have been developed, no one technique provides all the information which is needed to select a treatment. Angiography is very effective in locating lesions in the coronary vasculature, but provides little information concerning the nature of the lesion. To provide better characterization of the lesion(s), a variety of imaging techniques have been developed for providing a more detailed view of the lesion, including intravascular ultrasound (IVUS), angioscopy, laser spectroscopy, computed tomography (CT), magnetic resonance imaging (MRI), and the like. None of these techniques, however, is completely successful in determining the exact of the lesion. In particular, such techniques provide little information regarding whether the plaque is stable or unstable.

Plaques which form in the coronaries and other vessels comprise inflammatory cells, smooth muscles cells, cholesterol, and fatty substances, and these materials are usually trapped between the endothelium of the vessel and the underlying smooth muscle cells. Depending on various factors, including thickness, composition, and size of the deposited materials, the plaques can be characterized as stable or unstable. The plaque is normally covered by an endothelial layer. When the endothelial layer is disrupted, the ruptured plaque releases highly thrombogenic constituent materials which are capable of activating the clotting cascade and inducing rapid and substantial coronary thrombosis. Such rupture of an unstable plaque and the resulting thrombus formation can cause unstable angina chest pain, acute myocardial infarction (heart attack), sudden coronary death, and stroke. It has recently been proposed that plaque instability, rather than the degree of plaque build-up, should be the primary determining factor for treatment selection.

A variety of approaches for distinguishing stable and unstable plaque in patients have been proposed. Some of the proposals involve detecting a slightly elevated temperature within unstable plaque resulting from inflammation. Other techniques involve exposure of the plaque to infrared light. It has also been proposed to introduce radiolabeled materials which have been shown by autoradiography to bind to stable and unstable plaque in different ways. External detection of the radiolabels, however, greatly limits the sensitivity of these techniques and makes it difficult to determine the precise locations of the affected regions. Thus far, none of these technologies has possessed sufficient sensitivity or resolution necessary to reliably characterize the plaque at the cellular level in the intact animal or man.

In pending application no. 09/670,412 filed on Sep. 26, 2000, the inventor herein proposes the in situ detection of labeled markers within body lumens to provide information on proliferative conditions within the lumens. In particular, the use of radio labeled binding substances, such as low-density lipoproteins, cellular precursors, including proteins, nucleic acids, and the like were proposed to provide for targeted binding at the proliferative sites. Specific binding substances listed in the application were monocyte chemoattractant peptide 1(MCP1), Z2D3 antibody, and fluorodeoxyglucose.

For all of these reasons, it would be desirable to provide improved methods and apparatus for distinguishing between stable and unstable plaque within the coronary and other patient vasculature. It would be further desirable if such methods and techniques could be applied to characterizing lesions in other body lumens, which are associated with other disease and proliferative conditions. In particular, it would be desirable to provide a additional and specific binding substances which are capable of binding to proliferative sites within body lumens, such as regions of thrombus formation within blood vessels. While the binding substances listed in the pending patent application described above are to believed to be effective, it would none the less be desirable to provide additional binding substances having differing and/or improved binding profiles. The provision and use of additional binding substances permits both detection of conditions which might not be detectable with other binding substances as well as allows for the configuration of panels of binding substances for providing characterization and bioprofiling of thrombus, plaque, and other luminal conditions based on binding of two or more binding substances. At least some of these objectives will be met by the inventions described hereinafter.

2. Description of the Background Art

U.S. Pat. Nos. 6,171,577 and 5,968,477 described the preparation of radiolabeled annexins and their use for imaging thrombus in the vasculature. Stratton et al. (1995) Circulation 92:3113-3121, considers the use of radiolabeled annexin V for intra-arterial thrombus detection. The use of radiolabeled agents for detecting atherosclerotic lesions is described in the medical literature. See, for example, Elmaleh et al. (1998) Proc. Natl. Acad. Sci. USA 95:691-695; Vallabhajosula and Fuster (1997) J. Nucl. Med. 38:1788-1796); Demos et al. (1997) J. Pharm. Sci. 86:167-171; Narula et al. (1995) Circulation 92:474-484; and Lees et al. (1998) Arteriosclerosis 8:461-470. U.S. Pat. No. 4,660,563, describes the injection of radiolabeled lipoproteins into a patient where the lipoproteins are taken up into regions of arteriosclerotic lesions to permit early detection of those lesions using an external scintillation counter. U.S. Pat. No. 5,811,814, describes and intravascular radiation-detecting catheter. The catheter is used to locate tagged red blood cells that may accumulate, for example, in an aneurysm. U.S. Pat. No. 5,429,133, describes a laparoscopic probe for detecting radiation concentrated in solid tissue tumors. Miniature and flexible radiation detectors intended for medical use are produced by Intra-Medical LLC, Santa Monica, Calif. (www.intra-medical.com). See also U.S. Pat. Nos. 4,647,445; 4,877,599; 4,937,067; 5,510,466; 5,711,931; 5,726,153; and WO 89/10760.

### DETAILED DESCRIPTION OF THE INVENTION

Methods, systems, and kits, are provided for assessing characteristics of lesions and other target sites within body lumens, particularly atherosclerotic lesions within a patient's vasculature, including the coronary vasculature, peripheral vasculature, and cerebral vasculature. The present invention relies on introducing a labeled marker, typically a radiolabeled marker, to the patient in such a way that the marker localizes within the lesion or target site in some manner which enables or facilitates assessment of that target site. Introduction of the labeled marker can be systemic, e.g., by injection or infusion to the patient's blood circulation for evaluation of lesions in the vasculature or other body lumens. Alternatively, introduction of the labeled markers can be local, e.g., catheter delivery directly to a target site within a blood vessel or other body lumen. Moreover, the labeled marker could be introduced systemically and locally in various combinations. After introduction to the patient, the labeled marker is taken up by the lesion or other target site, and the amount of marker (accumulation), rate of uptake, distribution of marker, or other marker characteristics is then determined in order to facilitate or enable diagnosis or other evaluation of the lesion. In particular, according to the present invention, the amount, rate of uptake, and/or distribution of the marker at or near the lesion or other target site is measured in situ using a detector which has been introduced into the body lumen and positioned in a known or measurable relationship to the lesion or other target site.

The present invention in particular relies on annexin V (referred to herein generally as annexin) as the marker which localizes at a lesion or other target site within a blood vessel or other body lumen. Annexin V is a human protein (36 kD) of 319 amino acids. Annexin V binds with a high affinity to the phosphatidylserine moiety which is exposed on activated platelets present during thrombus formation within the vasculature. Advantageously, a local infusion of the annexin and nutrients may stabilize the lesion and bring the cells out of the stress (prothrombotic) state. The use of technetium 99 m-labeled annexin V for intra-arterial thrombus detection has been suggested in Stratton et al. (1995) supra. While the present invention will find particular use in the diagnosis of diseased lesions within the vasculature, most particularly in the diagnosis of coronary artery disease in the coronary vasculature, it will also be useful in a wide variety of other circumstances where uptake of a labeled substance can be related to diagnosis of a disease or other evaluation of a body lumen. For example, by introducing labeled annexin, various conditions related to excessive cellular proliferation can be assessed and monitored. For example, the presence or prognosis of various luminal cancers can be determined, such as cancer of the urinary bladder, colon cancer, esophageal cancer, prostate cancer (as well as benign prostate hyperplasia), lung cancer and other bronchial lesions, and the like, can be made.

The detection of the labeled annexin marker in situ within a body lumen has a number of significant advantages. Such in situ detection allows the detection of labels, such as visible light, fluorescence, luminescence, and the like, which cannot be detected externally. With tissue-penetrating labels, such as radioisotopic radiation, in situ detection is much more sensitive than external detection. This is particularly the case when lower energy (short-path length) radiation sources are used, such as beta (β) radiation, conversion electrons, and the like. Detection of lower energy radiation reduces the background which is observed when the tracer concentrates in an adjacent organ or tissue, and is usually not feasible with external detection which, for example, relies on the introduction gamma (γ) radiation-emitting labels and the use of gamma (γ) cameras. The present invention, however, is not limited to the use of beta (β) radiation, conversion electrons, and other short path length radiation, but instead may find use with all types of ionizing radiation under appropriate circumstances.

In situ detection also improves detection of both the position and distribution of labeled immobilized within the body lumen. It will be appreciated that the detectors can be configured and/or repositioned so that immobilized radiation and other labels can be determined with an accuracy of less than 5 mm, usually less than 3 mm, preferably less than 2 mm, and often less than 1 mm, along the axis of the body lumen. The ability to accurately locate a target site, such as a region of unstable plaque, a region of proliferating cells, or the like, can greatly facilitate subsequent treatment.

The labeled annexin marker will comprise at least two components, i.e., a detectable label and annexin which acts as a binding substance. The detectable label can be any natural or synthetic material which is capable of in situ detection using an intravascular catheter or other intraluminal detector. Particularly suitable are radiolabels comprising radionuclides which emit beta (β) radiation, conversion electrons, and/or gamma (γ) radiation. Presently preferred are radiolabels which emit primarily beta (β) radiation or conversion electrons which have a relatively short path length and permit more precise localization of the target site or material. By using detector(s) capable of quantifying both beta (β) and gamma (γ) radiation, it will be possible to gauge how close the detector is to the label based on the observed ratio of beta (β)/gamma (γ) radiation and the known emission characteristics of the label. That is, the relative decline in observed beta (β) radiation will include that the detector is further from the label.

In addition to radiolabels, the present invention can employ other visible markers including fluorescent labels, such as fluorescein, Texas Red, phycocyanin dyes, arylsulfonate cyanine dyes, and the like; chemiluminescent labels, and/or bioluminescent labels. The present invention can also employ passive labels which respond to interrogation in various ways. For example, the labels may comprise paramagnetic or superparamagnetic materials which are detected based on magnetic resonance. Alternatively, the labels may be acoustically reflective or absorptive, allowing detection by ultrasonic reflection. Further, the labels could be absorptive or reflective to infrared radiation, allowing detection by optical coherence tomography.

The labels will typically be bound, covalently or non-covalently, to the annexin binding substance. Specific labeled annexin substances and methods for their production are taught, for example, in Stratton et al (1995) supra as well as U.S. Pat. Nos. 6,171,577 and 5,968,477, the full disclosures of which are incorporated herein by reference.

In addition to the labeled annexin substances described above, the methods of the present invention may also use a second binding substance (other than annexin) bound to a detectable label. Such additional binding substances can be virtually any material which becomes incorporated into and/or bound to a desired intraluminal target site. For example, stressed macrophages could be stabilized by the introduction of a conjugation of annexin with fructose or other metabolic precursors to allow the cells to generate more ATP without the addition of oxygen.

Thus, in the case of intravascular detection and labeling of atherosclerotic lesions, the second binding substance may be a natural substance which becomes incorporated into the lesions, such as low-density lipoproteins or components thereof. In the case of excessive self-proliferation, the second binding substances can be a variety of cellular precursors, including proteins, nucleic acids, and the like. In addition to natural materials which become incorporated into a growing or proliferating target site, the second binding substances can be prepared or synthesized for specific binding to a target site at the target location. For example, antibodies can be prepared to a wide variety of vascular and non-vascular target sites. Additionally, in some cases, natural receptors and/or ligands will be available for particular target sites. For example, monocyte chemoattractant peptide 1 (MCP1) localizes on receptors upregulated by the macrophages in plaque. Other target substance in plaque include lectins whose receptors are upregulated on endothelial cells that overly the plaque. Antibodies such as Z2D3 (Khaw et al., Carrio et al., Narula et al.) localize on proliferating smooth muscle in the plaque. Another potential agent is fluorodeoxyglucose labeled with fluorine-18. This agent emits positions and is utilized as an energy substrate by macrophages and monocytes, and it has shown enhanced localization in experimental atherosclerosis models.

The label and annexin or second binding substance may be bound to each other in any conventional manner. Most commonly, moieties on the label and/or the binding substance will be derivitized to permit covalent attachment to the annexin or second binding substance. Covalent attachment will usually be direct, but in some cases may employ a linking member. Non-covalent attachment can employ a variety of non-covalent linkers, such as biotin, avidin, intermediate antibodies, receptors, ligands, and the like. A variety of suitable binding techniques are described in a review article in Nature Biotechnology (1999) Vol. 17, pages 849 and 850, the full disclosure of which is incorporated by reference.

A variety of suitable labeled markers have been proposed in the medical and scientific literature. See, for example, U.S. Pat. Nos. 4,647,445; 4,660,563; 4,937,067; 4,877,599; 5,510,466; 5,711,931; 5,726,153; and WO 89/10760. Each of these patent references is hereby incorporated in its entirety by reference.

An important aspect of the present invention is the ability to detect and/or image the label in situ after the label has localized in the blood vessel wall or other body lumen. Because the label binds to specific target materials within the body lumen, the pattern in which the label has localized will correspond to the pattern of the target material in the body lumen. Such separate detection may be performed simultaneously, sequentially, or in some combination thereof. For example, the annexin as well as certain second labeled binding substances, such as low-density lipoproteins, or a component thereof, will bind to atherosclerotic plaque which is actively growing or accumulating and therefore at risk of being unstable. The pattern of label(s) will thus correspond to the pattern of unstable plaque within the patient's vasculature.

Detection of the label and its pattern within the body lumen will be performed using an intraluminal detector, usually a detector capable of detecting ionizing radiation from a radioisotopic label within a particular distance of the label, as discussed in more detail below. The detector and catheter can be introduced into the body lumen by a variety of conventional techniques. For intravascular detectors the preferred techniques will be percutaneous, e.g., using a needle and sheath for introduction of a guidewire in a Seldinger access technique. Alternatively, surgical cutdowns can be used for accessing blood vessels, and a variety of other surgical and minimally invasive techniques can be used for introducing intraluminal detectors into other body lumens.

The nature of the label and characteristics of the detector will be selected so that an emitted signal from the label will be visible or detectable only within a particular distance of a detecting surface or element of the detector usually within 5 mm, preferably within 3 mm, and sometimes within 1 mm. That is, the detector will only have a limited range for viewing localized label so that background from label located remotely from the detector will not be detected. In this way, accurate positional detection of the label can be achieved. In a presently preferred embodiment, the label will emit beta (β) radiation or conversion electrons or low energy x-rays which have a very short path length. The sensitivity of the detector will then be selected so that the beta (β) radiation will be visible only over a very short distance, typically less than 3 mm, preferably less than 1 mm. Moreover, the detector may be configured so that its detector surface(s) or element(s) will be engaged directly against the wall of the blood vessel or other body lumen to enhance detection of the charged particle radiation.

In a particular aspect of the present invention, detection of the label will be performed over a minimum length of the body lumen in order to characterize variations in the luminal lesion over that length with the ability to distinguish lesions present at intervals of 3 mm. For example, in blood vessels, the present invention will usually be used to image over a vascular length of at least 30 mm, preferably at least 40 mm, and more preferably at least 50 mm. Such detection may be achieved by scanning a detector over the length within the blood vessel or other body lumen. Preferably, however, the detector can remain stationary within the lumen and have spatial resolution over the preferred minimum length set forth above without movement of the detector itself.

In addition to the minimum detection lengths set forth above, the detectors will preferably be isotropic over at least their circumference or periphery. Regardless of whether the detector is scanned or held stationary during detection, it will normally be preferred that detection of label over the entire circumference or periphery of the body lumen be performed. In other cases, however, it might be desired to perform a directional scan i.e., one where a particular radial sector of the body lumen wall is observed.

In some cases, it may be preferred to employ two or more labels (which may be an annexin only or on second binding substances) and to separately detect those labels in order to determine the special distribution of more than one material in the body lumen. For example, in addition to annexin which localized on activated platelets, plaques at different phases of development have varying degrees of smooth muscle proliferation (detectable with Z2D3 antibody localization), varying degrees of macrophage infiltration (detectable with MCP 1), varying levels of macrophage metabolism (detectable with the metabolic substrate FDG), and varying degrees of metalloproteinase activity (detectable with labeled antibodies specific for the metalloproteinase may be detected). Two or more parameters could be evaluated simultaneously if the radiopharmaceuticals carry radiolabels with substantially different energies or if one radionuclide has a substantially shorter half life than the other(s). Alternatively, labels having different natures, e.g., light emission, fluorescence emission, and/or radioisotopic radiation could be employed and detected simultaneous with minimum interference.

Detection of the localized annexin marker (either alone or in combination with a second or further marker) can provide useful information regarding a lesion or other structural condition of the body lumen. As described above, the present invention will permit determination of the axial and circumferential distribution of the target material within the body lumen. In the case of atherosclerotic lesions in a blood vessel, this information is particularly suitable for assessing the need for treatment as well as planning particular treatment modalities. In particular, the present inventor would allow the identification of relatively small lesions, e.g., with luminal blockage below 50%, which nonetheless are unstable and require immediate intervention. Conversely, larger lesions (above 50% occlusion) which are stable and less in need of immediate intervention can also be identified.

While the present invention is directed at intraluminal detection of marker(s), it may find use in combination with external detection of the same or other markers and/or external detection and imaging of the catheter which is being used for the intraluminal detection. External detection of immobilized markers may be useful for pre-positioning of the intraluminal detection catheter and/or for comparing information from different markers and targets (where the different markers may be bound to different binding substances having different specificities). External detection of the catheter will allow mapping of the vasculature or other luminal system. The position of the catheter can be detected fluoroscopically, by MRI, or otherwise, and the position of the internally detected lesions be noted on the external image or map which is created.

The methods of the present invention rely on the use of radiation detection devices comprising an elongate body, typically a catheter, and a radiation detector disposed on the elongate body. The catheter or other elongate body is configured to access the interior of a target body lumen, such as a blood vessel, a ureter, a urethra, an esophagus, a cervix, a uterus, a bladder, or the like. The radiation detector is capable of sensing radiation emitted into the body lumen and which is incident along the elongate body. In a first particular embodiment, the radiation detector will be capable of sensing radiation over a length of at least 3 cm, preferably at least 4 cm, and more preferably at least 5 cm. Optionally, the radiation detector will be capable of sensing radiation isotropically preferably being equally sensitive in all radial directions over the circumference of the elongate body.

In a second specific embodiment, the radiation detectors of the present invention will be capable of distinguishing radiation from at least two different radioactive labels with energies that differ by a threshold level.

In a third specific embodiment, the radiation detectors of the present invention will be capable of being axially translated within the body to sense radiation incident along the body over a length of at least 3 cm, preferably at least 4 cm, and more preferably at least 5 cm. Usually, such devices will comprise a catheter having an outside body which can remain stationary within a blood vessel and an internal detector which can be axially translated within the stationary body. Alternatively, the entire catheter may be translated within the lumen to cover the desired length.

Optionally, the catheters may comprise two or more different detection systems. Thus, in addition to the label detection system, the catheters might further indicate optical, ultrasonic, OCT, MR or other imaging systems. This will allow image information from the catheter to be “registered” or coordinated with the lesion characteristics also detected by the catheter. In some instances, it might be useful to provide for catheter-based excitation of a first or second label which has been immobilized at a target site.

The present invention still further comprises kits for identifying or assessing luminal lesions or other target sites. The kits will comprise a radiation detector configured to be introduced into a body lumen and instructions for use according to any of the methods described above.

Alternatively, kits according to the present invention may comprise a radiation detector configured to be introduced into a body lumen, a container for holding a reagent comprising a labeled annexin and optionally one or more additional substances capable of binding to a target material within the body lumen and a detectable label bound to the substance, and a package for holding the radiation detector and the containers together. The package may be any conventional package, such as a box, tray, tube, pouch, or the like. Instructions for use will typically be provided on a separate package insert, but in some cases may be printed in whole or in part on the packaging itself. Usually, the radiation detector will be maintained sterilely within the packaging.

The following examples are offered by way of illustration, not by way of limitation.

### EXPERIMENTAL

Twenty-one adult male rabbits (weighing 3-4 kg at the time of tracer administration) were each were fed a high cholesterol, high fat diet for one week. Following one week of feeding, the rabbits were heparinized, anesthetized with ketamine HCI (25 mg/kg, IM) and medetomidine (0.5 mg/kg, IM), and underwent a left carotid arteriotomy. A 3-4F Fogarty catheter was advanced through the arteriotomy under fluoroscopic guidance into the right iliac artery. The Fogarty balloon was inflated with 0.4-0.75 ml of contrast media (to assist with localization by fluoroscopy) and withdrawn to the level of diaphragm. The balloon was deflated, the catheter returned to the distal iliac under fluoroscopic guidance, and the process repeated. There were six repetitions of the procedure. The neck incision was closed, and the animals received Baytril (5 mg IM) for 3 days following surgery to minimize infection. The animals were allowed to recover for four additional weeks while the high fat/high cholesterol diet was continued. The rabbits were divided into seven groups of two each. On the day of study, a blood sample was drawn to measure lipids (cholesterol), and two groups of three animals received a single tracer dose intravenously via a marginal ear vein. The first group of three rabbits received annexin V-^{99m}Tc. The second group of three rabbits received albumin-¹²⁵I in the amounts shown in Table I below.

Two hours after the radiopharmaceutical was administered, the animals were injected with Evans blue to identify the areas of injury an re-endothelialized aorta and iliac vessel. Thirty minutes later, the animals were anesthetized with sodium pentothal (35 mg/kg, IV) and, a thoracotomy and a midline laporotomy incision were performed. The left ventricle was identified, a trocar placed in the chamber, and a cannula placed in the abdominal aorta. The rabbit was partially exsanguinated, followed by infusion of 100 mL lactated Ringer's solution, and subsequently perfused with 0.4% paraformaldehyde (PFA) for about 5 min at about 100 mmHg pressure.

Following euthanasia, the aorta was harvested, photographed (SONY Mavica) and autoradiographed (Molecular Dynamics). An aliquot of blood and an aliquot of the injected dose were included on the plate to permit quantification. The autoradiographs were compared to the Evans Blue stained specimen to determine where the tracer had localized with reference to the injury. A ratio between uptake in the injured and uninjured iliac vessel was determined (average uptake ratio) using analysis option of ‘Image Quest¹.’ Additional ratios were created using the average counts in regions of interest placed over the area with peak uptake on the autoradiograph with each agent, and a ratio of peak uptake to the activity in an aliquot blood were determined. A summary of the results are listed in Table I.

TABLE I

| | Average | Peak | | | |
|---|---|---|---|---|---|
| Agent | Uptake | Uptake | Peak to | Figure | |
| (activity | Ratio | Ratio | Blood Ratio | of Merit | Chemistries |
| administered) | (AUR) | (PUR) | (PBR) | (FOM) | Glu/Chol |
| Albumin | 3.0 | 5.2 | .3 | 14 | 134/3650 |
| (150 uci) | | | | | |
| Annexin | 8.4 | 15.0 | 4.5 | 1701 | 125/3543 |
| (3 mci) | | | | | |

To simplify the process of integrating the information, a Figure of Merit was calculated, using the formula:

FOM=3(AUR) (PUR) (PBR)

Albumin, the control substance, has a substantial differential between the areas of arterial lesions and the uninjured vessel. Quantitatively, the injured iliac vessel had 3 fold greater activity than the uninjured site (animals 1 and 3), but in animal 2 this ration was 1:1. Peak lesion to normal ration was 5.1, but the lesion to blood ration was 0.3. Annexin had an average of 8.4 fold greater uptake in the injured site, with a peak uptake of 15 fold the uninjured zone. Peak lesion uptake to blood ratio was 4.5. There was remarkable highlighting of the perimeter of the re-endothelized regions. In addition, there was a striking correlation between lesion sites and localization of annexin on the autoradiograph.

While the above is a complete description of the preferred embodiments of the invention, various alternatives, modifications, and equivalents may be used. Therefore, the above description should not be taken as limiting the scope of the invention which is defmed by the appended claims.

What is claimed is:

1. A method for identifying or assessing a luminal lesion, said method comprising:
introducing a first detectable marker comprising annexin to a body lumen, wherein said annexin localizes at a lesion comprising a material which preferentially binds or incorporates the annexin;
introducing a detector into the body lumen; and
detecting the detectable marker which has accumulated at the site of the lesion using the detector.

2. A method as in claim 1, wherein introducing the detectable marker comprises introducing a radioactive marker

3. A method as in claim 2, wherein introducing the radioactive marker comprises introducing a radioisotope which emits beta (β) radiation.

4. A method as in any of claims 1 to 3, wherein introducing the detectable marker comprises systemic introduction of the marker.

5. A method as in any of claims 1 to 3, wherein introducing the detectable marker comprises introducing locally through the detector.

6. A method as in any of claims 1 to 3, wherein introducing the detectable marker comprises systemic injection into the patient's vasculature.

7. A method as in claim 1, wherein introducing the detector comprises percutaneous introduction of a catheter into the body lumen, wherein the catheter comprises the detector.

8. A method as in claim 1, wherein introducing the detector comprises surgically accessing the body lumen to create an access passage and advancing the catheter through the passage into the body lumen, wherein the catheter comprises the detector.

9. A method as in claim 1, wherein detecting the localized marker comprises detecting marker within 5 mm of a detection surface of the detector.

10. A method as in claim 1, wherein the marker emits beta (β) radiation and the detector detects beta (β) radiation.

11. A method as in any of claims 1, 9, and 10, wherein detecting the localized marker comprises scanning a segment of the body lesion having a length of at least 3cm

12. A method as in claim 11, wherein the scanned length is at least 4 cm.

13. A method as in claim 11, wherein scanning the segment comprises positioning a detector having a detection surface and scanning while the detector remains stationary in the body lumen.

14. A method as in claim 11, wherein scanning the segment comprises positioning a detector and repositioning the detector to scan the entire segment.

15. A method as in any of claims 1, 9, and 10, wherein detecting the localized marker comprises isotropically detecting marker disposed about a periphery of the body lumen.

16. A method as in claim 1, further comprising:
introducing a second detectable marker to the body lumen, wherein said second detectable marker localizes at a lesion comprising a second material which preferentially binds the second detectable marker; and
detecting the second detecting marker.

17. A method as in claim 16, wherein the second detectable marker is detected using the same detector as detected the first detectable marker.

18. A method as in claim 16, wherein the second detectable marker is detected using a second detector.

19. A method for assessing the stability of an intravascular lesion, said method comprising:
introducing radiolabeled annexin into a blood vessel, wherein the annexin is selectively incorporated into plaque which is at increased risk of rupture;
introducing a detector into the blood vessel; and
determining to what degree the radiolabeled annexin has been incorporated into a lesion, whereby an assessment of lesion stability may be made.

20. A method as in claim 19, wherein introducing the radiolabeled annexin comprises introducing a annexin complex radioisotope which emits beta (β) radiation.

21. A method as in either of claims 19 and 20, wherein introducing radiolabeled annexin comprises systemic introduction of the radiolabeled annexin.

22. A method as in either of claims 19 and 20, wherein introducing the radiolabeled annexin comprises introducing locally through the detector.

23. A method as in either of claims 19 and 20, wherein introducing the radiolabeled annexin comprises systemic injection into the patient's vasculature.

24. A method as in claim 19, wherein introducing the detector comprises percutaneous introduction of a catheter into the blood vessel, wherein the catheter comprises the detector.

25. A method as in claim 19, wherein introducing the detector comprises surgically accessing the blood vessel to create an access passage and advancing the catheter through the passage into the blood vessel, wherein the catheter comprises the detector.

26. A method as in claim 19, wherein determining the degree of incorporation comprises detecting radiolabeled annexin within 5 cm of a detection surface of the detector.

27. A method as in claim 26, wherein the radiolabeled substance emits beta (β) radiation and the detector detects beta (β) radiation.

28. A method as in any of claims 19, 26, and 27, wherein determining the degree of incorporation comprises scanning a segment of the body lesion having a length of at least 3 cm.

29. A method as in claim 28, wherein the scanned length is at least 4 cm.

30. A method as in claim 28, wherein scanning the segment comprises positioning a detector having a detection surface and scanning while the detector remains stationary in the body lumen.

31. A method as in claim 28, wherein scanning the segment comprises positioning a detector and repositioning the detector to scan the entire segment.

32. A method as in any of claims 19, 26, and 27, wherein determining the degree of incorporation comprises isotropically detecting marker disposed about a periphery of the body lumen.

33. A method as in claim 19, further comprising:
introducing a second radiolabeled substance to the blood vessel, wherein said second radiolabeled substance localizes at a lesion comprising a second material which preferentially binds the lesion; and
detecting the second radiolabeled substance.

34. A method as in claim 33, wherein the second radiolabeled substance is detected using the same detector as detected the first radiolabeled substance

35. A method as in claim 33, wherein the second radiolabeled substance is detected using a second detector.

36. A kit comprising:
a radiation detector configured to be introduced into a body lumen; and
instructions for use according to either of claims 1 and 19

37. A kit comprising:
a radiation detector configured to be introduced into a body lumen;
a container holding labeled annexin a capable of binding to a target within the body lumen; and
a package for holding the radiation detector and the container.
